SEQUENCE LISTING (5 sequences)

SEQ ID NO: 1 - protein, 976 aa, Homo sapiens [sequence omitted]

SEQ ID NO: 2 - DNA, 30 bp, Homo sapiens
  Primer
aagaagagatggtacctcgaggggtgaccc

SEQ ID NO: 3 - DNA, 33 bp, Homo sapiens
  Primer
ctgcttcgcggccgcgttaactcttctcaacca

SEQ ID NO: 4 - DNA, 20 bp, Homo sapiens
  Primer
agctcgtttagtgaaccgtc

SEQ ID NO: 5 - DNA, 20 bp, Homo sapiens
  Primer
gtcagacaaaatgatgcaac

(19) United States
(12) Patent Application Publication
(10) Pub. No.: US 2005/0054617 A1
(43) Pub. Date: Mar. 10, 2005

(54) Use of potent, selective and non toxic c-kit inhibitors for treating mastocytosis

(76) Inventors: Alain Moussy, Paris (FR); Jean-Pierre Kinet, Lexington, MA (US)

Correspondence Address: SUGHRUE MION, PLLC, 2100 PENNSYLVANIA AVENUE, N.W., SUITE 800, WASHINGTON, DC, 20037, US

(21) Appl. No.: 10/482,179

(22) PCT Filed: Jun. 28, 2002
(86) PCT No.: PCT/IB02/03303

Related U.S. Application Data
(60) Provisional application No. 60/301,406, filed on Jun. 29, 2001.

(51) Int. Cl. A61K 31/66; A61K 31/517; A61K 31/505

(52) U.S. Cl. 514/102; 514/275; 514/414; 514/266.1

(57) ABSTRACT

The present invention relates to a method for treating mastocytosis comprising administering a tyrosine kinase inhibitor to a human in need of such treatment, more particularly a non-toxic, selective and potent c-kit inhibitor, wherein said inhibitor is unable to promote death of IL-3 dependent cells cultured in presence of IL-3. The invention also contemplates a composition for topical application comprising said inhibitor for treating category I mastocytosis.

The present invention relates to a method for treating mastocytosis comprising administering a tyrosine kinase inhibitor to a human in need of such treatment, more particularly a non toxic, potent and selective c-kit inhibitor, wherein said inhibitor is unable to promote death of IL-3 dependent cells cultured in presence of IL-3. The invention also contemplates a composition for topical application comprising said inhibitor for treating category I mastocytosis.

Mast cells (MC) are tissue elements derived from a particular subset of hematopoietic stem cells that express CD34, c-kit and CD13 antigens (Kirshenbaum et al, Blood. 94: 2333-2342, 1999 and Ishizaka et al, Curr Opin Immunol. 5: 937-43, 1993). Immature MC progenitors circulate in the bloodstream and differentiate in tissues. These differentiation and proliferation processes are under the influence of cytokines, one of utmost importance being Stem Cell Factor (SCF), also termed Kit ligand (KL), Steel factor (SL) or Mast Cell Growth Factor (MCGF). SCF receptor is encoded by the protooncogene c-kit, that belongs to type III receptor tyrosine kinase subfamily (Boissan and Arock, J Leukoc Biol. 67: 135-48, 2000). This receptor is also expressed on others hematopoietic or non hematopoietic cells. Ligation of c-kit receptor by SCF induces its dimerization followed by its transphosphorylation, leading to the recruitement and activation of various intracytoplasmic substrates. These activated substrates induce multiple intracellular signaling pathways responsible for cell proliferation and activation (Boissan and Arock, 2000). Mast cells are characterized by their heterogeneity, not only regarding tissue location and structure but also at the functional and histochemical levels (Aldenborg and Enerback., Histochem. J. 26: 587-96, 1994 ; Bradding et al. J Immunol. 155: 297-307, 1995 ; Irani et al, J Immunol. 147: 247-53, 1991; Miller et al, Curr Opin Immunol. 1: 637-42, 1989 and Welle et al, J Leukoc Biol. 61: 233-45, 1997).

Indeed, at least three different subtypes of mast cells exist in humans, that differ by their morphological appearance, their tissue location, their biochemical content and their reactivity towards various compounds. These three different subtypes of mast cells are distinguished on the basis of their content of neutral proteases. Mast cells containing only tryptase (T) are termed MCT, while MC containing tryptase and chymase (C) are known as MCTC. The main differences between these two major subsets of human MC are presented in Table I. Additionally, a minor population of mast cells expresses only chymase, but not tryptase, and are named MCC (Li et al, J Immunol. 156: 4839-44, 1996). Concerning their functions, besides their role already largely explored as cells involved in immediate hypersensitivity, recent studies have been able to show that mast cells possess two major physiological properties as antigen presenting cells, and as elements highly involved in the anti-infectious defense of the organism (Abraham and Arock, Semin Immunol. 10: 373-381, 1998 ; Arock and Abraham, Immunol. 66: 6030-4, 1998; Galli et al, Curr Opin Immunol. 11: 53-59, 1999).

Mastocytosis, that represents an heterogeneous group of relatively rare diseases, is characterized by accumulation of MC in various tissues, and can be found isolated or sometimes associated with others hematological malignancies in humans. Today, regarding its biological features, mastocytosis (with or without myeloid accompanying disorders) is considered to be an hematologic disease. Although the initial events leading to mastocytosis are not yet unraveled, alterations of the c-kit gene have been described in a significant proportion of the patients. Particularly interesting are acquired mutations resulting in a constitutively activated receptor, possibly involved in the increased numbers of MC in tissues. For this reason, future strategies might be envisaged to target specifically the mutated c-kit and/or its intracellular signaling.

Mastocytosis are a very heterogeneous group of disorders characterized by an abnormal accumulation of mast cells in different tissues, mainly in the skin and the bone marrow, but also in spleen, liver, lymph nodes, and the gastrointestinal tract, depending on the nature of the disease. They can affect humans of either sex at any age. Neoplasms of MC can be acute or chronic. Acute mast cell neoplasms are designated as MC leukemia. Chronic mast cell neoplasms may be localized or generalized. Cutaneous mastocytosis is the commonest localized neoplasm and is often found in children in which it often remits and never relapses. Mastocytosis are usually acquired diseases, but some rare familial cases have been described.

With regard to the extreme heterogeneity of mast cell neoplasms, it is important to classify these diseases. One of the most used classification is the one by Metcalfe (Metcalfe, J Invest Dermatol. 96: 2S-4S, 1991) that distinguishes four categories of mastocytosis:

The category I is composed by two subcategories (IA and IB). Category IA is made by diseases in which mast cell infiltration is strictly localized to the skin. This category represents the most frequent form of the disease and includes: i) urticaria pigmentosa, the most common form of cutaneous mastocytosis, particularly encountered in children, ii) diffuse cutaneous mastocytosis, iii) solitary mastocytoma and iv) some rare subtypes like bullous, erythrodermic and teleangiectatic mastocytosis. These forms are characterized by their excellent prognosis with spontaneous remissions in children and a very indolent course in adults. Long term survival of this form of disease is generally comparable to that of the normal population and the translation into another form of mastocytosis is rare. Category IB is represented by indolent systemic disease (SM) with or without cutaneous involvement. These forms are much more usual in adults than in children. The course of the disease is often indolent, but sometimes signs of aggressive or malignant mastocytosis can occur, leading to progressive impaired organ function.

The category II includes mastocytosis with an associated hematological disorder, such as a myeloproliferative or myelodysplastic syndrome, or acute leukemia. These malignant mastocytosis does not usually involve the skin. The progression of the disease depends generally on the type of associated hematological disorder that conditiones the prognosis.

The category III is represented by aggressive systemic mastocytosis in which massive infiltration of multiple organs by abnormal mast cells is common. In patients who pursue this kind of aggressive clinical course, peripheral blood features suggestive of a myeloproliferative disorder are more prominent. The progression of the disease can be very rapid, similar to acute leukemia, or some patients can show a longer survival time.

Finally, the category IV of mastocytosis includes the mast cell leukemia, characterized by the presence of circulating mast cells and mast cell progenitors representing more than 10% of the white blood cells. This entity represents probably the rarest type of leukemia in humans, and has a very poor prognosis, similar to the rapidly progressing variant of malignant mastocytosis. Mast cell leukemia can occur either de novo or as the terminal phase of urticaria pigmentosa or systemic mastocytosis.

Since categories II and III do not differ prognostically, the classification of Metcalfe can be further simplified as shown in Table I, according to the recommendations of Horny et al (Horny et al, Am J Surg Pathol. 22: 1132-40, 1998).

TABLE I

| Localized (category I) | Generalized (categories II, III, IV) |
|---|---|
| Cutaneous mastocytosis | Systemic mastocytosis (with or without |
| | cutaneous involvement) |
| Solitary mastocytoma | Indolent |
| Urticaria pigmentosa | Aggressive |
| | Mast cell leukemia |

Clinical symptoms of mastocytosis result from the release of chemical mediators of mast cells and the infiltration of different organs by mast cells. Regarding infiltration of various organs by these elements and their clinical consequences, as well as the main adverse effects of mast cell-derived mediators, findings are the following:

Peripheral Blood

In patients with an indolent cutaneous form, the peripheral blood is normal in the vast majority of cases. In patients with an indolent form of systemic disease, the peripheral blood is most often normal, but a minority of patients has neutrophilia, eosinophilia, basophilia, monocytosis, thrombocytosis or lymphocytosis (Travis et al, Cancer. 62: 965-72, 1988; Horny et al, Br J Haematol. 76: 186-93, 1990). A very small number of circulating mast cells may be present. In case of aggressive disease, the majority of patients have neutrophilia, many have eosinophilia, basophilia or monocytosis, and a minority has thrombocytosis. By contrast, some patients may present cytopenias, particularly anemia and thrombocytopenia, but leucopenia and neutropenia may also be found. Some patients have circulating mast cells, usually in small numbers.

In mast cell leukemia, the peripheral blood shows mast cells in numbers varying from patient to patient (Torrey et al, Am J Hematol. 34: 283-6, 1990 ; Baghestanian et al, Leukemia. 10: 159-66, 1996). These mast cells are often immature or abnormal with hypogranularity or nuclear lobulation (Torrey et al, 1990). These neoplastic mast cells may sometimes be so cytologically atypical that it is difficult to distinguish them from abnormal basophils.

Bone Marrow

Bone marrow infiltration by MC characterizes most of the cases of systemic mastocytosis. MC are not always increased when the sample examined is a bone marrow aspirate. Indeed, due to fibrosis provoked by their proliferation, they can be under-evaluated. Besides, the bone marrow cellularity may remain normal in indolent SM, with only a small number of mast cells with nearly normal appearance, while bone marrow samples of patients with an aggressive course are likely to show hypercellularity, with granulocytic hyperplasia and large numbers of MC with frequent cytological atypia (Pari et al, Recenti Prog Med. 90: 169-72, 1999). In some cases, features of myelodysplasia can be found (Valent et al, Blood. 84: 4322-32, 1994). In mast cell leukemia, the bone marrow is hypercellular and largely infiltrated by abnormal mast cells (Le Cam et al, Ann Dermatol Venereol. 124: 621-2, 1997).

The marrow biopsy is abnormal in the vast majority of cases of SM The most common finding is focal infiltration by mast cells, randomly distributed or in paratrabecular and perivascular areas (Pari et al, 1999 ; Genovese et al, Int J Clin Lab Res. 25: 178-88, 1995). Diffuse interstitial infiltrates of MC are less common. There is usually a dense network of reticulin fibers associated with the infiltrate, and even osteosclerosis (Alexander et al, Acta Haematol. 74: 108-10, 1985).

Mast cells in bone narrow aspirates or in trephine biopsies may sometimes be difficult to characterize by the use of classical staining procedures, due to their atypia, especially in mast cell leukemia. In these cases, the use of immunocytochemistry with monoclonal antibodies specific to MC tryptase is very useful in confirming the MC nature of the infiltrate.

Infiltration in Other Tissues

In systemic mastocytosis with multiple organs involvement, infiltrates of mast cells in tissues are formed by clusters of mast cells in portal areas of liver, perifollicular of spleen, perivascular of skin or in sinus of lymph nodes (Metcalfe, J Invest Dermatol. 96: 45S-46S, 1991). Hepatomegaly and splenomegaly have been observed in 50% of patients with systemic mastocytosis, resulting in infiltration of liver and spleen by mast cells (Pauls et al, Arch Intem Med. 159: 401-5, 1999). Nodal lesions, poorly documented in the literature, seem to be more common in malignant forms or associated with a hematological disorder. Bone lesions are often clinically silent. However, if symptoms are present, they usually refer to lytic lesions, osteoporosis or marrow fibrosis. Then, radiological examination often shows diffuse abnormalities, more rarely focal or mixed (Weide et al, Ann Hematol. 72: 41-3, 1996; Grieser et al, Lancet. 350: 1103-4, 1997).

Adverse Effects of Mast Cell-Derived Mediators

Many symptoms can be related to the release of mediators such as histamine and prostaglandins by infiltrating MC. Gastrointestinal symptoms are frequent in patients with systemic mast cell disease and are generally represented by nausea, vomiting, diarrhea, abdominal pain and alcohol intolerance (Pari et al, 1999 ; Miner et al, J Invest Dermatol. 96: 40S-43S, 1991). Other clinical signs can be related to mediators released by mast cells in the skin: skin flushing, pruritus, heat and cold intolerance or in the general circulation: palpitations, shortness of breath, lipothymy, decrease in blood pressure, coagulation deffect as the consequence of the release of heparin and, occasionally, syncope and shock (Bain et al, Br J Haematol. 106: 9-17, 1999 ; Soter, J Invest Dermatol. 96: 32S-38S, 1991). In addition, one can notice than even patients having only a cutaneous form of the diseaese may present systemic symptoms, due to the activity of mediators released in the bloodstream from the original lesion. Symptomatic release of MC granules may be precipitated by emotional disturbance, exertion, exposure to heat, exposure to alcohol, aspirin, opiates, anticholinergics, non-steroidal anti-inflammatory drugs and contrast media (Valent, Wien Klin Wochenschr. 108: 385-97, 1996).

Molecular Genetic Lesions in Mastocytosis

Differentiation, survival and proliferation of MC depend greatly on SCF (Torrey et al, 1990). The receptor for SCF is c-kit, encoded by the protooncogene c-kit; it belongs to type III receptor tyrosine kinase subfamily (Baghestanian et al, 1996). Numerous studies have been performed regarding the neoplastic mechanism of mastocytosis, searching for genetic abnormalities of c-kit (mutation, deletion). The existence of such abnormalities was suggested because they were previously found in rodent or human leukemic MC lines. In human mastocytosis, mutations of c-kit have been described in vivo in various forms of mastocytosis (cutaneous mastocytosis, systemic indolent or systemic aggressive mastocytosis). Among the mutations found, the most common is the activating mutation Asp to Val at codon 816. For example, this mutation has been identified in mast cells from patients with aggressive systemic mastocytosis (Pari et al, 1999), with indolent cutaneous mastocytosis in adult (Valent et al, 1994) or in child (Le Cam et al, 1997). In addition, one report has described a mutation in the juxtamembrane domain of c-kit (Val to Gly at codon 560) in human mastocytosis (Valent et al, 1994). By contrast, the role of this mutation at codon 560 has been evoked in some cases of gastrointestinal tumors (GIST) (Genovese et al, 1995). Moreover, other point mutations in the c-kit gene have been reported by Pignon et al (Alexander et al, 1985 and Metcalfe, 1991) at codon 820, in the tyrosine kinase domain, resulting in substitution of Asp for Gly in MC from a patient with an aggressive mastocytosis, and by Longley et al (Pauls et al, 1999) in codon 816, causing substitution of tyrosine or phenylalanine for aspartate in child with sporadic systemic mastocytosis or cutaneous mastocytosis, and also in codon 839 with substitution of lysine for glutamic acid in child with sporadic indolent urticaria pigmentosa. Furthermore, Longley et al (Pauls et al, 1999) have showed that the point mutations in 816 caused spontaneous phosphorylation of c-kit, whereas c-kit with the mutation at the 839 was not autophosphorylated or phosphorylated after exposure to exogenous SCF, and even inhibited the autophosphorylation of c-kit mutated at the 816 position.

Finally, the situation observed in the few familial cases reported in the literature seems to be different from that of sporadic diseases, since no mutations of c-kit were found in a very limited number of patients (Pauls et al, 1999).

In conclusion, as concerns the structure of the c-kit gene, human mastocytosis can be divided in three groups:
mastocytosis with activating mutations, mainly in codon 816, representing probably most of the cases of adult SM;
mastocytosis with inactivating mutation, such as in codon 839, particularly encountered in children with urticaria pigmentosa;
mastocytosis without any c-kit mutation, covering the rare cases of familial mastocytosis.

The proposed treatments of mastocytosis (inhibition of the release of MC mediators or inhibition of the deleterious effects of such mediators) are symptomatic treatments aim at interfering with the adverse effects induced by the abnormal production of mediators by mast cells. The main molecules used are H1 and H2 antihistamines (Gasior-Chrzan et al, Dermatology. 184: 149-52, 1992). H1 antihistamines are usually administered against pruritus, flushing, whereas H2 antihistamines are used to treat gastritis and peptic ulcer. Other molecules like corticosteroids may be necessary in the case of severe cutaneous symptoms (Burrall et al, Chronic urticaria., West J Med. 152: 268-76, 1990). Also, anticholinergics are administered to treat diarrheas and headache (Valent, 1996). Disodium cromoglycate, an inhibitor of mast cell degranulation, is used to mitigate respiratory symptoms (Martinez-Orozco et at Med Clin (Barc). 78: 77, 1982). Acute cardiovascular collapse may require adrenaline and intravenous fluids. Patients who are prone to such attacks should carry adrenaline for self-administration (Bain et al, 1999). It seems likely that biphosphonates would be useful in patients with osteoporosis and pathological fractures (Pari et al, 1999). Such treatments alleviate the symptoms associated with mastocytosis but do not constitute long term treatments of this disease.

Therefore, the general goal of the present invention is to provide a solution for inhibiting mast cells proliferation, which is the cause of mastocytosis.

In this regard, interferons INFα and INFγ are used in the art associated or not with corticosteroids (Pari et al, 1999). The concept of the use of interferons is based. on the fact that aggressive mastocytosis are similar to myeloproliferative syndromes such as chronic myeloid leukemia in which INFα can induce, in some cases, loss of Philadelphia chromosome. Fiehn et al, Eur J Clin Invest. 25: 615-8, 1995 have described a case of systemic mastocytosis with infiltration of bone marrow, skin, and gastric mucosa in a 81 years old woman; she has been treated with INFγ and has presented an improvement of the clinical situation and gastrointestinal and hematological signs. A recurrence has been noted after four months, concomitantly to the appearance of circulating antibody to interferon A similar case has been reported by Delaporte et al, Br J Dermatol. 132: 479-82, 1995 where association between INFα and corticosteroids during ten months has improved clinical situation without recurrence at arrest to interferon. Furthermore, some anaphylactic reactions to interferon have been noticed (Pardini et al, Acta Haematol. 85: 220, 1991), so it must be started at a low dose. These results are quite exciting, but this treatment by interferon does not correct the initial abnormality, i.e.; the presence of activating mutations of c-kit in abnormal MC.

Indeed, since it appears now that the major forms of the disease are associated with activating mutations of the c-kit in its tyrosine kinase domain, aiming at blocking this abnormal tyrosine kinase activity could be a challenge in the next years for near future therapeutic strategies of systemic mastocytosis. For instance, similar therapeutics have been considered in chronic myelogenous leukemia with the development of a Bcr-Abl inhibitor to block signaling transduction pathways that causes the abnormal proliferation of the granulocytic serie found of this malignant disease at its chronic stage (Boissan and Arock, Leukoc Biol. 67: 135-48, 2000).

In this way, Ma et al have tested in vitro several indolinones derivatives (SU4984, SU6663, SU6577 and SU5614), that are specific tyrosine kinase inhibitors, and found that some of these compounds can inhibit the constitutively activated c-kit mutants (Ma et al, J Invest Dermatol. 114: 392-4, 2000). However, it is shown in this publication that among the compound tested, only SU6577 at 40 μM could substantially reduce receptor phosphorylation of the D816 mutant activated c-kit. This compound is also active on c-kit wild, but at a 40 μM concentration, the problem is that the activity of SU6577 on the D816 mutant might result from toxicity. A lack of specificity on c-kit versus other tyrosine kinases would render such a compound inadequate for therapeutic purposes.

Thus, the aim of the invention is to provide compounds that are selective, potent but also non toxic inhibitors of c-kit.

Many different compounds have been described as tyrosine kinase inhibitors, but none of these compounds, however, have been demonstrated to selectively inhibit activated c-kit, while being unable to promote death of IL-3 dependent cells cultured in presence of IL-3, resulting in a lower toxicity.

Furthermore, mast cells are implicated in tumoral pathologies, particularly in systemic mastocytosis that are hematological diseases similar to myeloproliferative syndromes. Of interest, mutations of c-kit have been described in vivo in different forms of mastocytosis, and occur in the intracytoplasmic tail of this receptor, mainly in its phosphotransferase domain. According to the position of the mutation, its effect on mast cell proliferation appears to be different. Indeed, these mutations can be found either in aggressive diseases or in indolent mastocytosis. c-kit mutations can also be found in mastocytosis associated with others malignant hemopathies, or less frequently in isolated hemopathies such as acute myeloid leukemia and myeloproliferative or myelodysplastic syndromes.

While some compounds may efficiently inhibit a given mutant c-kit, they may not inhibit different mutants responsible for c-kit activation or SCF activated c-kit. Another problem for physicians is therefore to have at their disposal a general inhibitor of c-kit acting on activated c-kit whatever the activation is, i.e mutation or SCF.

The present invention, directed to a method for treating mastocytosis whatever the cause of c-kit activation is, i.e SCF activation or mutation activation, comprising administering compounds that are inhibitors of SCF activated c-kit and/or constitutively activated c-kit, also provides a solution to this problem.

### DESCRIPTION

The present invention relates to a method for treating mastocytosis comprising administering a tyrosine kinase inhibitor to a mammalian in need of such treatment, wherein said inhibitor is unable to promote death of IL-3 dependent cells cultured in presence of IL-3.

Tyrosine kinase inhibitors are selected for example from bis monocyclic, bicyclic or heterocyclic aryl compounds (WO 92/20642), vinylene-azaindole derivatives (WO 94/14808) and 1-cycloproppyl-4-pyridyl-quinolones (U.S. Pat. No. 5,330,992), Styryl compounds (U.S. Pat. No. 5,217,999), styryl-substituted pyridyl compounds (U.S. Pat. No. 5,302,606), seleoindoles and selenides (WO 94/03427), tricyclic polyhydroxylic compounds (WO 92/21660) and benzylphosphonic acid compounds (WO 91/15495), pyrimidine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834,504, U.S. Pat. No. 5,883,116, U.S. Pat. No. 5,883,113, U.S. Pat. No. 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940) and aryl and heteroaryl quinazoline (U.S. Pat. No. 5,721,237, U.S. Pat. No. 5,714,493, U.S. Pat. No. 5,710,158 and WO 95/15758).

Preferably, said tyrosine kinase inhibitors are non-toxic, selective and potent c-kit inhibitors. Such inhibitors can be selected from the group consisting of indolinones, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, seleoindoles, selenides, tricyclic polyhydroxylic compounds and benzylphosphonic acid compounds.

Among preferred compounds, it is of interest to focus on pyrimidine derivatives such as N-phenyl-2-pyrimidine-amine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834,504), U.S. Pat. No. 5,883,116, U.S. Pat. No. 5,883,113, U.S. Pat. No. 5, 886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940), 4-amino-substituted quinazolines (US 3,470,182), 4-thienyl-2-(1H)quinazolones, 6,7-dialkoxyquinazolines (U.S. Pat. No. 3,800,039), aryl and heteroaryl quinazoline (U.S. Pat. No. 5,721,237, U.S. Pat. No. 5,714,493, U.S. Pat. No. 5,710,158 and WO 95/15758), 4-anilinoquinazoline compounds (U.S. Pat. No. 4,464,375), and 4-thienyl-2-(1H)-quinazolones (U.S. Pat. No. 3,551,427).

So, preferably, the invention relates to a method for treating mastocytosis comprising administering a non toxic, potent and selective c-kit inhibitor to a mammalian in need of such treatment, selected from the group consisting of
pyrimidine derivatives, more particularly N-phenyl-2-pyrimidine-amine derivatives.
indolinone derivatives, more particularly pyrrol-substituted indolinones,
monocyclic, bicyclic aryl and heteroaryl compounds,
and quinazoline derivatives,
   wherein said inhibitor is unable to promote death of IL-3 dependent cells cultured in presence of IL-3.

In another embodiment, c-kit inhibitors as mentioned above are inhibitors of activated c-kit. In frame with the invention, the expression “activated c-kit” means a constitutively activated-mutant c-kit including at least one mutation selected from point mutations, deletions, insertions, but also modifications and alterations of the natural c-kit sequence (SEQ ID N°1). Such mutations, deletions, insertions, modifications and alterations can occur in the transphosphorylase domain, in the juxtamembrane domain as well as in any domain directly or indirectly responsible for c-kit activity. The expression “activated c-kit” also means herein SCF-activated c-kit. Preferred and optimal SCF concentrations for activating c-kit are comprised between 5.10⁻⁷ M and 5.10⁻⁶ M, preferably around 2.10⁻⁶ M. In a preferred embodiment, the activated-mutant c-kit in step a) has at least one mutation proximal to Y823, more particularly between amino acids 800 to 850 of SEQ ID No1 involved in c-kit autophosphorylation, notably the D816V, D816Y, D816F and D820G mutants. In another preferred embodiment, the activated-mutant c-kit in step a) has a deletion in the juxtamembrane domain of c-kit. Such a deletion is for example between codon 573 and 579 called c-kit d(573-579). The point mutation V559G proximal to the juxtamembrane domain c-kit is also of interest.

In this regard, the invention contemplates a method for treating mastocytosis comprising administering to a mammalian in need of such treatment a compound that is a selective, potent and non toxic inhibitor of activated c-kit obtainable by a screening method which comprises
a) bringing into contact (i) activated c-kit and (ii) at least one compound to be tested; under conditions allowing the components (i) and (ii) to form a complex,
b) selecting compounds that inhibit activated c-kit,
c) testing and selecting a subset of compounds identified in step b), which are unable to promote death of IL-3 dependent cells cultured in presence of IL-3.

This screening method can further comprise the step consisting of testing and selecting a subset of compounds identified in step b) that are inhibitors of mutant activated c-kit (for example in the transphosphorylase domain), which are also capable of inhibiting SCF-activated c-kit wild.

Alternatively, in step a) activated c-kit is SCF-activated c-kit wild.

A best mode for practicing this method consists of testing putative inhibitors at a concentration above 10 μM in step a). Relevant concentrations are for example 10, 15, 20, 25, 30, 35 or 40 μM.

In step c), IL-3 is preferably present in the culture media of IL-3 dependent cells at a concentration comprised between 0.5 and 10 ng/ml, preferably between 1 to 5 ng/ml.

Examples of IL-3 dependent cells include but are not limited to:
cell lines naturally expressing and depending on c-kit for growth and survival. Among such cells, human mast cell lines can be established using the following procedures:
normal human mast cells can be infected by retroviral vectors containing sequences coding for a mutant c-kit comprising the c-kit signal peptide and a TAG sequence allowing to differentiate mutant c-kits from c-kit wild expressed in hematopoetic cells by means of antibodies.

This technique is advantageous because it does not induce cellular mortality and the genetic transfer is stable and gives satisfactory yields (around 20%). Pure normal human mast cells can be routinely obtained by culturing precursor cells originating from blood obtained from human umbilical vein. In this regard, heparinated blood from umbilical vein is centrifuged on a Ficoll gradient so as to isolate mononucleated cells from other blood components. CD34+ precursor cells are then purified from the isolated cells mentioned above using the immunomagnetic selection system MACS (Miltenyi biotech). CD34+ cells are then cultured at 37° C. in 5% CO₂ atmosphere at a concentration of 10⁵ cells per ml in the medium MCCM (α-MEM supplemented with L-glutamine, penicillin, streptomycin, 5 10⁻⁵ M β-mercaptoethanol, 20% veal fetal serum, 1% bovine albumin serum and 100 ng/ml recombinant human SCF. The medium is changed every 5 to 7 days. The percentage of mast cells present in the culture is assessed each week, using May-Grünwal Giemsa or Toluidine blue coloration. Anti-tryptase antibodies can also be used to detect mast cells in culture. After 10 weeks of culture, a pure cellular population of mast cells (<98%) is obtained.

It is possible using standard procedures to prepare vectors expressing c-kit for transfecting the cell lines established as mentioned above. The cDNA of human c-kit has been described in Yarden et al., (1987) EMBO J.6 (11), 3341-3351. The coding part of c-kit (3000 bp) can be amplified by PCR and cloned, using the following oligonucleotides

| | |
|---|---|
| sens | |
| 5′AAGAAGAGATGGTACCTCGAGGGGTGACCC3′ | (SEQ ID No 2) |
| antisens | |
| 5′CTGCTTCGCGGCCGCGTTAACTCTTCTCAACCA3′ | (SEQ ID No 3) |

The PCR products, digested with Not1 and Xho1 , has been inserted using T4 ligase in the pFlag-CMV vector (SIGMA), which vector is digested with Not1 and Xho1 and dephosphorylated using CIP (Biolabs). The pFlag-CMV-c-kit is used to transform bacterial clone XL1-blue. The transformation of clones is verified using the following primers:

| | |
|---|---|
| sens | |
| 5′AGCTCGTTTAGTGAACCGTC3′, | (SEQ ID No 4) |
| antisens | |
| 5′GTCAGACAAAATGATGCAAC3′. | (SEQ ID No 5) |

Directed mutagenesis is performed using relevant cassettes is performed with routine and common procedure known in the art.

The vector Migr-1 (ABC) can be used as a basis for constructing retroviral vectors used for transfecting mature mast cells. This vector is advantageous because it contains the sequence coding for GFP at the 3′ and of an IRES. These features allow to select cells infected by the retrovirus using direct analysis with a fluorocytometer. As mentioned above, the N-terminal sequence of c-kit c-DNA can be modified so as to introduce a Flag sequence that will be useful to discriminating heterogeneous from endogenous c-kit.

Other IL-3 dependent cell lines that can be used include but are not limited to:
BaF3 mouse cells expressing wild-type or mutated form of c-kit (in the juxtamembrane and in the catalytic sites) are described in Kitayama et al, (1996), Blood 88, 995-1004 and Tsujimura et al, (1999), Blood 93, 1319-1329.
IC-2 mouse cells expressing either c-kit^{WT} or c-kit^{D814Y} are presented in Piao et al, (1996), Proc. Natl . Acad. Sci. U.S.A. 93, 14665-14669.

IL-3 independent cell lines are:
HMC-1, a factor-independent cell line derived from a patient with mast cell leukemia, expresses a juxtamembrane mutant c-kit polypeptide that has constitutive kinase activity (Furitsu T et al, J Clin Invest. 1993;92:1736-1744 ; Butterfield et al, Establishment of an immature mast cell line from a patient with mast cell leukemia. Leuk Res. 1988;12:345-355 and Nagata et al, Proc Natl Acad Sci U.S.A. 1995;92:10560-10564).
P815 cell line (mastocytoma naturally expressing c-kit mutation at the 814 position) has been described in Tsujimura et al, (1994), Blood 83, 2619-2626.

The extent to which component (ii) inhibits activated c-kit can be measured in vitro or in vivo. In case it is measured in vivo, cell lines expressing an activated-mutant c-kit, which has at least one mutation proximal to Y823, more particularly between amino acids 800 to 850 of SEQ ID No1 involved in c-kit autophosphorylation, notably the D816V, D816Y, D816F and D820G mutants, are preferred.

Example of cell lines expressing an activated-mutant c-kit are as mentioned above.

In another preferred embodiment, the method further comprises the step consisting of testing and selecting compounds capable of inhibiting c-kit wild at concentration below 1 μM. This can be measured in vitro or in vivo.

Therefore, compounds are identified and selected according to the method described above are potent, selective and non-toxic c-kit wild inhibitors.

Alternatively, the screening method according to the invention can be practiced in vitro In this regard, the inhibition of mutant-activated c-kit and/or c-kit wild can be measured using standard biochemical techniques such as immunoprecipitation and western blot. Preferably, the amount of c-kit phosphorylation is measured.

In a still further embodiment, the invention contemplates a method for treating mastocytosis as depicted above wherein the screening comprises:
a) performing a proliferation assay with cells expressing a mutant c-kit (for example in the transphosphorylase domain), which mutant is a permanent activated c-kit, with a plurality of test compounds to identify a subset of candidate compounds targeting activated c-kit, each having an IC50<10 μM, by measuring the extent of cell death,
b) performing a proliferation assay with cells expressing c-kit wild said subset of candidate compounds identified in step (a), said cells being IL-3 dependent cells cultured in presence of IL-3, to identify a subset of candidate compounds targeting specifically c-kit,
c) performing a proliferation assay with cells expressing c-kit, with the subset of compounds identified in step b) and selecting a subset of candidate compounds targeting c-kit wild, each having an IC50<10 μM, preferably an IC50<1 μM, by measuring the extent of cell death.

Here, the extent of cell death can be measured by 3H thymidine incorporation, the trypan blue exclusion method or flow cytometry with propidium iodide. These are common techniques routinely practiced in the art.

Therefore, the invention embraces the use of the compounds defined above to manufacture a medicament for treating mastocytosis in mammalian, especially in human and in dogs.

The pharmaceutical compositions utilized in this invention may be administered by any number of routes including, but not limited to, oral, intravenous, intramuscular, intra-arterial intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient. Suitable excipients are carbohydrate or protein fillers, such as sugars, including lactose, sucrose, mannitol, or sorbitol, starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

The pharmaceutical composition may be provided as a salt and can be formed with many acids, including but not limited to, hydrochloric, sulfuric, acetic, lactic, tartaric, malic, and succine, acids, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

Pharmaceutical compositions suitable for use in the invention include compositions wherein c-kit inhibitors are contained in an effective amount to achieve the intended purpose. The determination of an effective dose is well within the capability of those skilled in the art. A therapeutically effective dose refers to that amount of active ingredient, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio of toxic to therpeutic effects is the therapeutic index, and it can be expressed as the ratio, LD50/ED50. Pharmaceutical compositions which exhibit large therapeutic indices are preferred. As mentioned above, a tyrosine kinase inhibitor and more particularly a c-kit inhibitor according to the invention is unable to promote death of IL-3 dependent cells cultured in presence of IL-3.

In addition, the invention relates to a method as defined above for treating category I, II, III and IV mastocytosis in human and any symptom associated with category I, II, III and IV mastocytosis. More specifically, the method according to the invention is useful for treating urticaria pigmentosa, diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, and mast cell leukemia.

The diagnosis of mastocytosis is mainly based on histological criterias and allow to assess what would be the best inhibitor on a case to case basis for a given patient. Indeed, with the method according to the invention, it is now possible to treat patients with appropriate inhibitors, within the appropriate formulation. For example, for category I matocytosis, a SCF-activated c-kit inhibitor administered with a topical composition is more suitable. Regarding category II, III and IV matocytosis, mutant activated c-kit inhibitors as defined above are more suitable. It should be mentioned that the invention also provides with compounds that are general activated c-kit inhibitors that can be used for treating any form of the disease.

The clinical suspicion of mastocytosis should be confirmed by histologic examination, especially of skin and bone marrow. Stains such as tuoluidine blue can be used to identify mast cells by staining their metachromatic granules. Also, the chloroacetate-esterase reaction can complete staining. In addition, immunocytochemistry for tryptase is useful to confirm the nature of the cellular infiltrate. Finally, the diagnostic can be helped by the use of the immunophenotyping of MC in bone marrow aspirate. Indeed, normal as well as mastocytosis-related mast cells strongly express CD117 antigen (Arber et al, Hum Pathol. 29: 498-504, 1998 ; Escribano et al, Cytometry. 30: 98-102, 1997), and some antigens not found on normal MC can be aberrantly expressed by neoplastic mast cells, such as CD2, CD25 and CD35 (Escribanoet al, Blood. 91: 2731-6, 1998, Ormerod et al, British Journal of Dermatology. 122: 737-44, 1990). In addition, recent findings have shown that the CD69 activation antigen is overexpressed on MC from patients with systemic mastocytosis, as compared to normal mast cells (Diaz-Agustin et al, Br J Haematol. 106: 400-5, 1999).

Biochemical determination of mast cell mediators can also help to diagnosis of mastocytosis: histamine level in blood and urine, metabolites of prostaglandin D2 and of histamine in the urine are increased in most cases of SM, as well as the level of tryptase in blood (Hogan and Schwartz, Methods 13: 43-52, 1997 ; Van Gysel et al, J Am Acad Dermatol. 35: 556-8, 1996 ; Morrow et al, J Invest Dermatol. 104: 937-40, 1995; Marone et al, Chem Immunol. 62: 1-21, 1995). However, with these tests, some false positive (allergy) or false negative (mastocytosis without mediator release) may exist. Standard molecular biology techniques based on PCR should are also contemplated for precisely determining the activating mutation in a given patient. Probes and primers can be designed so as to be specific to such mutations analysis and are derived from SEQ ID N°1 segments and complementary sequences thereof (see Table II below).

TABLE 2
Major mutations of c-kit described in patients with isolated mastocytosis.
UP: Urticaria pigmentosa; SM: Systemic mastocytosis; CM: Cutaneous mastocytosis
in which the type is not stated precisely. Sol M: Solitary mastocytoma; CMd: Cutaneous
mastocytosis diffuse; Adult sp: Adult sporadic; Adult fam: Adult familial. nt: activity
of the mutation has not been tested

| | | | Consequence | Phenotype of | | |
|---|---|---|---|---|---|---|
| | | C-kit | of the | the | | |
| Patients | N^{o} | mutation | mutation | disease | Tissue | References |
| Adult sp | 1 | CTG-862 CTC | silent | Sol M | Focal | Longley et al, N Engl |
| | | | | | bone | J Med. 328: 1302-7, |
| | | | | | | 1993 |
| Adult sp | 1 | D816V | activating | UP + SM | skin + spleen | Morrow et al, 1995 |
| Adult sp | 1 | D816V | activating | UP | skin | |
| Adult sp | 5/5 | D816V | nt | UP + SM | skin | Morrow et al, 1995 |
| Adult sp | 1/1 | D816V | nt | UP | skin | |
| Child sp | 11/11 | neg | — | UP or CMd or | skin | |
| | | | | Sol M | | |
| Adult sp | 1/4 | V560G | nt | UP + SM | skin | Morrow et al, 1995 |
| Adult sp | 1/4 | V560G | nt | UP | skin | |
| Adult sp | 1 | D820G | unknown | SM | bone | Costa et al, J Exp |
| | | | | | marrow | Med. 183: 2681-6, |
| | | | | | | 1996 |
| Child sp | 1 | D816V | nt | UP | skin | Granerus et al, |
| | | | | | | Inflamm Res. 48: 75-80, |
| | | | | | | 1999 |
| Adult sp | 8/1 | D816V | activating | CM | skin | Longley et al, Ann |
| Adult sp | 1 | D816V | activating | SM | skin | Med. 26: 115-6, |
| Child sp | 3/1 | E839K | inactivating | UP | skin | 1994 |
| Child | 1 | neg | no mutation | UP | skin | |
| fam | 3/1 | neg | no mutation | UP | skin | |
| Adult | 1 | D816Y | activating | CM + SM | skin | |
| fam | 1/1 | D816F | activating | CM + SM | skin | |
| Child sp | 2/2 | D816V | activating | CM + SM | skin | |
| | 2/1 | | | | | |
| | 1 | | | | | |
| | 1/1 | | | | | |
| | 1 | | | | | |
| | 1/1 | | | | | |
| | 1 | | | | | |

Consequently, in yet another embodiment, the method of treatment according to the invention comprises the step of diagnosing the category of mastocytosis in a given individual and administering the suitable c-kit inhibitor in the suitable form.

As far as dog mastocyoma is concerned, Spontaneous mast cell tumors (MCT) are the most common malignant neoplasm in the dog, representing between 70% and 21% of all canine tumors, an incidence much higher than that found in humans. These tumors often behave in an aggressive manner, metastasizing to local lymph nodes, liver, spleen, and bone marrow. Whereas point mutations in the kinase domain of c-kit leading to tyrosine phosphorylation in the absence of ligand binding have been identified in some human patients with various forms of mastocytosis, it has been recently demonstrated that c-kit derived from canine MCT possessed novel mutations consisting of tandem duplications involving exons 11 and 12 (Valent, 1996). It was also showed that such duplication, detected in a canine mastocytoma cell line, was associated with constitutive phosphorylation of c-kit protein. We have found in connection with the invention that these mutations may contribute to the development or progression of canine MCT, and that compounds aiming at blocking specifically such mutations might be useful, if non-toxic, in the treatment of MCT. Therefore, tyrosine kinase inhibitors and more particularly non toxic c-kit inhibitors as defined above are good candidate compounds for treating this disease in dogs.

For treating category II, III and IV mastocytosis, oral, intravenous and intramuscular route of administration are preferred.

In a still further embodiment, the invention is directed to a composition comprising a tyrosine kinase inhibitors, more particularly an activated c-kit inhibitor as well as a non toxic, potent and selective c-kit inhibitor as defined above for topical application Such composition is adapted for treating skin disorders associated with mastocytosis in human, notably cutaneous mastocytosis including urticaria pigmentosa, diffuse cutaneous mastocytosis, solitary mastocytoma and bullous, erythrodermic and teleangiectatic mastocytosis.

The compositions according to the invention may be presented in all forms normally used for topical application, in particular in the form of a gel, paste, ointment, cream, lotion, liquid suspension aqueous, aqueous-alcoholic or, oily solutions, or dispersions of the lotion or serum type, or anhydrous or lipophilic gels, or emulsions of liquid or semi-solid consistency of the milk type, obtained by dispersing a fatty phase in an aqueous phase or vice versa, or of suspensions or emulsions of soft, semi-solid consistency of the cream or gel type, or alternatively of microemulsions, of microcapsules, of microparticles or of vesicular dispersions to the ionic and/or nonionic type. These compositions are prepared according to standard methods.

The composition according to the invention comprises any ingredient commonly used in dermatology and cosmetic. It may comprise at least one ingredient selected from hydrophilic or lipophilic gelling agents, hydrophilic or lipophilic active agents, preservatives, emollients, viscosity enhancing polymers, humectants, surfactants, preservatives, antioxidants, solvents, and fillers, antioxidants, solvents, perfumes, fillers, screening agents, bactericides, odor absorbers and coloring matter.

As oils which can be used in the invention, mineral oils (liquid paraffin), vegetable oils (liquid fraction of shea butter, sunflower oil), animal oils, synthetic oils, silicone oils (cyclomethicone) and fluorinated oils may be mentioned. Fatty alcohols, fatty acids (stearic acid) and waxes (paraffin, carnauba, beeswax) may also be used as fatty substances.

As emulsifiers which can be used in the invention, glycerol stearate, polysorbate 60 and the PEG-6/PEG-32/glycol stearate mixture are contemplated.

As hydrophilic gelling agents, carboxyvinyl polymers (carbomer), acrylic copolymers such as acrylate/alkylacrylate copolymers, polyacrylamides, polysaccharides such as hydroxypropylcellulose, clays and natural gums may be mentioned, and as lipophilic gelling agents, modified clays such as bentones, metal salts of fatty acids such as aluminum stearates and hydrophobic silica, or alternatively ethylcellulose and polyethylene may be mentioned.

As hydrophilic active agents, proteins or protein hydrolysates, amino acids, polyols, urea, allantoin, sugars and sugar derivatives, vitamins, starch and plant extracts, in particular those of Aloe vera may be used.

As lipophilic active, agents, retinol (vitamin A) and its derivatives, tocopherol (vitamin E) and its derivatives, essential fatty acids, ceramides and essential oils may be used. These agents add extra moisturizing or skin softening features when utilized.

In addition, a surfactant can be included in the composition so as to provide deeper penetration of the ingredients and of the tyrosine kinase inhibitor.

Among the contemplated ingredients, the invention embraces penetration enhancing agents selected for example from the group consisting of mineral oil, water, ethanol, triacetin, glycerin and propylene glycol; cohesion agents selected for example from the group consisting of polyisobutylene, polyvinyl acetate and polyvinyl alcohol, and thickening agents.

Chemical methods of enhancing topical absorption of drugs are well known in the art. For example, compounds with penetration enhancing properties include sodium lauryl sulfate (Dugard, P. H. and Sheuplein, R. J., “Effects of Ionic Surfactants on the Permeability of Human Epidermis: An Electrometric Study,” J. Ivest. Dermatol., V.60, pp. 263-69, 1973), lauryl amine oxide (Johnson et. al., U.S. Pat. No. 4,411,893), azone (Rajadhyaksha, U.S. Pat. Nos. 4,405,616 and 3,989,816) and decylnethyl sulfoxide (Sekura, D. L. and Scala, J., “The Percutaneous Absorption of Alkylmethyl Sulfides,” Pharmacology of the Skin, Advances In Biolocy of Skin, (Appleton-Century Craft) V. 12, pp. 257-69, 1972). It has been observed that increasing the polarity of the head group in amphoteric molecules increases their penetration-enhancing properties but at the expense of increasing their skin irritating properties (Cooper, E. R. and Berner, B., “Interaction of Surfactants with Epidermal Tissues: Physiochemical Aspects,” Surfactant Science Series, V. 16, Reiger, M. M. ed. (Marcel Dekker, Inc.) pp. 195-210, 1987).

A second class of chemical enhancers are generally referred to as co-solvents. These materials are absorbed topically relatively easily, and, by a variety of mechanisms, achieve permeation enhancement for some drugs. Ethanol (Gale et. al., U.S. Pat. No. 4,615,699 and Campbell et. al., U.S. Pat. Nos. 4,460,372 and 4,379,454), dimethyl sulfoxide (U.S. Pat. Nos. 3,740,420 and 3,743,727, and U.S. Pat. No. 4,575,515), and glycerine derivatives (U.S. Pat. No. 4,322,433) are a few examples of compounds which have shown an ability to enhance the absorption of various compounds.

The invention is also directed to a method for treating category IV mastocytosis including mast cell leukemia, comprising administering a tyrosine kinase inhibitor, preferably a c-kit inhibitor as defined above and a compound selected from 2-Chloro-2′-desoxyadenosine and analogs thereof to a mammalian in need of such treatment, wherein said inhibitor is unable to promote death of IL-3 dependent cells cultured in presence of IL-3. In this regard, the invention also contemplates a product comprising at least one tyrosine kinase inhibitor, preferably a c-kit inhibitor as defined above, and at least one compound selected from 2-Chloro-2′-desoxyadenosine and analogs thereof for a separate, sequential or simultaneous use for treating category IV mastocytosis including mast cell leukemia.

2-Chloro-2′-desoxyadenosine (2-CDA), Cladribine, Merck Index (12th ed.) #2397 has the following formula:

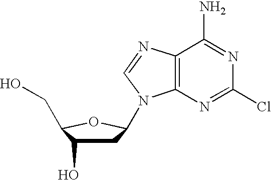

Regarding systemic forms of mastocytosis, especially category III mastocytosis, the invention also relates to a method as mentioned above, comprising administering a tyrosine kinase inhibitor, preferably a c-kit inhibitor and IFNα to a human in need of such treatment, wherein said inhibitor is unable to promote death of IL-3 dependent cells cultured in presence of IL-3. In this regard, the invention also contemplates a product comprising at least one tyrosine kinase inhibitor, preferably a c-kit inhibitor as defined above, and IFNα for a separate, sequential or simultaneous use for treating systemic forms of mastocytosis, especially category III mastocytosis.

Utility of the invention will further ensues from the detailed description below.

### EXAMPLE 1

### Molecular Genetic Lesions in Mastocytosis.

Patient Findings

Differentiation, survival and proliferation of MC depend on cytokines, one of utmost importance being SCF (Costa et al, 1996). The receptor for SCF is c-kit, encoded by the protooncogene c-kit; it belongs to type III receptor tyrosine kinase subfamily (Flanagan et al, Cell. 64: 1025-35, 1991). With the development of recent data, the two main factors that could be involved in the abnormal proliferation of mast cells in mastocytosis appear to be SCF and its specific receptor, c-kit. In fact, several authors have investigated the role of SCF and c-kit in the pathogenesis of this disease. Besides, it is difficult to determine exactly whether the mastocytosis are tumoral pathologies or reactional disorders (Longley et al, Ann Med. 26: 115-6, 1994). The hyperplastic hypothesis of mastocytosis has been related by Longley et al, N Engl J Med. 328: 1302-7, 1993 in some cases of cutaneous mast cell disease, i.e.; the majority of benign mastocytosis. They have found increased levels of the soluble form of SCF in the skin of patients with indolent cutaneous mastocytosis. In these cases, no mutation of the SCF gene was identified, suggesting an aberrant metabolism of SCF. However, this mechanism is since poorly documented.

By contrast, numerous studies have been performed regarding the neoplastic mechanism of mastocytosis, searching for genetic abnormalities of c-kit (mutation, deletion) that converts it into an oncoprotein able to induce oncogenic transformation of mast cells. The existence of such abnormalities was suggested because they were previously found in rodent or human leukemic MC lines. Indeed, different somatic point mutations have been described in rodent and in human cell lines (see below), which contribute to SCF independent activation of c-kit and probably to neoplastic proliferation of mast cells. In most cases, these mutations are found in the catalytic domain of the c-kit and are activating.

In human mastocytosis, a number of studies have been performed to elucidate whether mutations of c-kit are associated with different clinical forms of mast cell diseases. Indeed, mutations of c-kit have been described in vivo in various forms of mastocytosis (cutaneous mastocytosis, systemic indolent or systemic aggressive mastocytosis). Among the mutations found, the most common is the activating mutation Asp to Val at codon 816. For example, this mutation has been identified in mast cells from patients with aggressive systemic mastocytosis (Longley et al, Nat Genet. 12: 312-4, 1996), with indolent cutaneous mastocytosis in adult (Buttner et al, J Invest Dermatol. 111: 1227-31, 1998) or in child (Nagata et al, Int Arch Allergy Immunol. 113: 184-6, 1997). By contrast, only one report has described the mutation Val to Gly at codon 560 in human mastocytosis. Indeed, Buttner et al (Buttner et al, 1998) have found this mutation in two of four lesional skin samples from adult mastocytosis; as this latter mutation has not been evidenced in any other study performed in patients, its reality remains to be confirmed. Nevertheless, the role of this mutation has been evoked in some cases of gastrointestinal tumors (GIST) carrying c-kit mutations in the juxtamembrane domain (Hirota et al, Science. 279: 577-80, 1998). In addition, other point mutations in the c-kit gene have been reported by Pignon et al (Pignon et al, Hematol Cell Ther. 39: 114-6, 1997 ; Pignon et al, Br J Haematol. 96: 374-6, 1997) at codon 820, in the tyrosine kinase domain, resulting in substitution of Asp for Gly in MC from a patient with an aggressive mastocytosis, and by Longley et al (Longley et al, Proc Natl Acad Sci U.S.A. 96: 1609-1614, 1999) in codon 816, causing substitution of tyrosine or phenylalanine for aspartate in child with sporadic systemic mastocytosis or cutaneous mastocytosis, and also in codon 839 with substitution of lysine for glutamic acid (c-kit^{E839}K) in child with sporadic indolent urticaria pigmentosa. Furthermore, Longley et al, Proc Natl Acad Sci USA. 96: 1609-1614, 1999 have showed that the mutations c-kit^{D816F} and c-kit^{D816Y}, as shown previously for c-kit^{D816V}, caused spontaneous phosphorylation of c-kit, whereas c-kit^{E839K} was not autophosphorylated or phosphorylated after exposure to exogenous SCF, and even inhibited the autophosphorylation of c-kit mutated at the 816 position. Given these data, the mutation at the 839 position could be termed as “inactivating”.

Interestingly, a very recent report has analyzed the distribution of the Asp816Val mutation among hematopoietic lineages by examination of cells bearing differentiation markers for myelomonocytic cells as well as T and B lymphocytes, in both peripheral blood and bone marrow obtained from patients with mastocytosis (Akin et al, Exp Hematol. 28: 140-7, 2000). In this study, the mutation was detectable by RT-PCR in at least one cell lineage in the bone marrow in 7 of 7 patients examined and in the peripheral blood of 11 of 11 adult patients with urticaria pigmentosa and indolent disease. The mutation was identified most frequently in B cells and myeloid cells. Flow cytometric analysis demonstrated that the differentiated cells expressing mutated c-kit were negative for surface c-kit. These results are consistent with the conclusion that the c-kit Asp816Val mutation occurs in an early progenitor cell, and not in mature mast cells since it is carried by myelomonocytic cells, T cells, and B cells in addition to MC.

In addition, the same activating point mutations in codon 816 of the c-kit gene have been described not only in patients with isolated mastocytosis but also in mastocytosis with an associated hematological disorder, such as a myeloproliferative or myelodysplastic syndrome, or acute leukemia (Boissan and Arock, Leukoc Biol. 67: 135-48, 2000).

Contrasting with the activating or inactivating mutations described above, some point mutations may be silent mutations and probably inconsequential. For instance, Nagata et al, Proc Natl Acad Sci USA. 92: 10560-4, 1995 have observed a single base change in a patient with a solitary mastocytoma (CTG to CTC at codon 862); both codons CTG and CTC encoding leucin. This silent mutation is probably not involved in the appearance of the disease, suggesting that this solitary mastocytosis could occur via abnormalities others than c-kit mutations.

Finally, the situation observed in the few familial cases reported in the literature seems to be different from that of sporadic diseases. Indeed, Longley et al, Proc Natl Acad Sci USA. 96: 1609-1614, 1999 have reported three patients, members of a kindred with familial cutaneous mastocytosis in a serie of 25 patients with mastocytosis: one child and two adults. In these three patients, no mutations of c-kit were found, suggesting that c-kit mutations are not involved in the physiopathology of familial mastocytosis.

In conclusion, as concerns the structure of the c-kit gene, human mastocytosis can be divided in three groups: a first group of mastocytosis with activating mutations, representing probably most of the cases of adult SM, a second group of mastocytosis with inactivating mutation, particularly encountered in children with urticaria pigmentosa and, finally, a third group of mastocytosis without any c-kit mutation, covering the rare cases of farmilial mastocytosis. A summary of these various findings is presented in Table III below.

TABLE 3
Abnormalities of the c-kit structure found in patients with mastocytosis
associated with others hematological disorders, or in patients with hematological
disorders not involving the mast cell lineage. SM: Systemic mastocytosis. PBMC:
Peripheral blood mononuclear cells; BMC: Bone marrow cells.

| Patients | | | | |
|---|---|---|---|---|
| Age | N^{o} | C-kit | Phenotype of the disease | Tissue |
| adult | 4 | D816V | SM with an associated myclofibrosis or | PBMC |
| | | | myelodysplastic syndrome | |
| adult | 3 | D52N | chronic myelogenous leukemia or | PBMC or |
| | | | primitive myelofibrosis | BMC |
| unknown | 1 | D816V | AML2 | BMC |
| child | 1 | D816V | bullous mastocytosis with an associated | BMC |
| | | | myeloproliferative syndrome | |
| adult | 1 | D816V | SM with AML4 | BMC |
| unknown | 7 | D816V | SM with an associated myeloproliferative | PBMC |
| | | | or myelodysplastic syndrome, or | |
| | | | hypereosinophilic syndrome | |
| adult | 1 | D816Y | SM with AML2 | BMC |
| unknown | 7 | deletion-insertion | AML with inv (16) | BMC |
| | | at exon 8 encoding | | |
| | | the fifth Ig-like | | |
| | | domain | | |
| unknown | 1 | mutationVal530Ile | AML with t(8; 21) | BMC |

In Vitro Data

Most of the present knowledge concerning the consequences of c-kit mutations in hematopoiesis and mast cell proliferation and activation has been obtained using various rodent or human cell lines bearing one or the other mutation. The primary goal of these in vitro studies was to demonstrate that c-kit mutations are sufficient by themselves to induce the abnormal proliferation of MC observed during mastocytosis in patients. Data presented here will show that this crucial question remains partly unresolved today, since they were principally obtained using cell lines and animal models. As a consequence, they might not reflect the precise situation encountered in humans.

At this time, four tumoral mast cell lines have been used to explore the consequences of the mutations in the c-kit gene. These mast cell lines are:—P815 and FMA3, two mouse mastocytoma cell lines, in which mutations cause, in codon 814, the substitution of Tyr for Asp in the phosphotransferase domain (Tsujimura et al, Blood. 83: 2619-26, 1994) and, in codons 573 to 579, deletion of seven amino acids in the juxtamembrane domain (Tsujimura et al, Blood. 87: 273-83, 1996) respectively.—RBL-2H3, a rat mast cell leukemia cell line, in which mutation cause, in codon 817, substitution of Tyr for Asp in the phosphotransferase domain (Tsujimura et al, Int Arch Allergy Immunol. 106: 377-85, 1995).—HMC1, the only mast cell line of human origin derived from a patient with a mast cell leukemia, in which two mutations have been identified: one, in codon 560 in the juxtamembrane of c-kit causing substitution of Val to Gly and another, in codon 816 in the tyrosine-kinase domain inducing substitution of Asp to Val (Furitsu et al, Journal of Clinical Investigation. 92: 1736-44, 1993).

Oncogenic potential of c-kit was primarily studied in these cell line models (P-815, FMA3, RBL-2H3 and HMC1). In these four mast cell tumors, c-kit was found constitutively phosphorylated on tyrosine and activated, inducing cell proliferation in the absence of SCF. Nevertheless, the different genetic abnormalities encountered in these cell lines have not the same biological effects. According to Furitsu et al, c-kit transforming activity is weaker with the mutation in position 560 than with the mutation in position 816 in HMC1 (Furitsu et al, 1993). Furthermore, excepted for the deletion of seven amino acids in FMA3, a somatic point mutation in the gene encoding c-kit, resulting in most cases into a change of a single amino acid, is enough to cause c-kit dysregulation. Moreover, amino acid substitution occurs in each species at equivalent codon.

In the view to understand the role of the point mutation in codon 814 in the catalytic domain of c-kit (an equivalent of the codon 816 in human c-kit), Piao et al, Blood. 87: 3117-23, 1996 have studied the biologic effects of the mutation, after its transfection into IC2 cells, an IL-3 dependent mast cell line that does not express endogenous wild type c-kit (WT). They have obtained three major data:—the mutant was phosphorylated on tyrosine residues in the absence of SCF,—the IC2 cells that express the mutant proliferated for more than 4 weeks in the absence of any growth factors and formed SCF independent colonies in vitro, and finally,—injection of IC2 cells that express the mutant c-kit into syngenic DBA/2 mice induced the development of hepatic mastocytomas in all the mice injected. These observations clearly demonstrated that the expression of this mutant in position 814 is sufficient to confer tumorigenic potential to IC2 cells.

Different molecular dysfunctions related to c-kit mutations have been described. In fact, it has been revealed that these mutations alter different aspects of c-kit metabolism concerning dimerization, signaling, enzyme expression, and internalization. These changes could explain oncogenic activation of c-kit.

Tsujimura et al (Tsujimura et al, Blood. 87: 273-83, 1996 ; Tsujimura et al, Pathol Int. 46: 933-8, 1996) and Kitayama et al (Kitayama et al, Blood. 85: 790-8, 1995.) have performed cross linking analysis of various c-kit receptors, wild type and mutated variants, to determine whether the constituvely activated c-kit leads to receptor dimerization or not, in the absence of SCF. For this, they have respectively studied four forms of c-kit: c-kit^{WT} (wild type), c-kit^{d(573-579)} (c-kit with a deletion from codon 573 to 579), c-kit^{V559G} (Val to Gly in codon 559), c-kit^{D814V} (Asp to Val in codon 814). These forms were introduced in Ba/F3 cells. They found that an activating deletion such as c-kit^{d(573-579)}, or an activating mutation, such as the c-kit^{V559G}, that take place in the juxtamembrane domain are able to induce a constitutive dirnerization of c-kit in the absence of SCF activation, whereas an activating mutation such as c-kit^{D814V} in the tyrosine kinase domain causes constitutive activation without dimerization. According to the authors, in the first case, a c-kit conformation change could induce its dimerization in the absence of SCF. Nevertheless, in the second case, the point mutation in the catalytic domain could trigger stimulation signaling by autophosphorylation without c-kit dimerization. However, more recently, Tsujimura et al, Blood. 93: 1319-29, 1999 have presented data indicating that c-kit^{D814V} devoid of extracellular domain, coimmunoprecipitated with full length wild type receptor or c-kit^{W42}, a dominant negative receptor. These authors proposed that self-association of c-kit^{D814V} might result from the mutation itself by creating a novel receptor self-association domain.

In addition, Piao, X et al, Proc Natl Acad Sci USA. 93: 14665-9, 1996 have reported signaling alterations through the c-kit^{D814Y} in the murine mast cell line IC2, as compared to wild-type c-kit. Indeed, in IC2 cells expressing c-kit^{D814Y}, they have detected not only the phosphorylation of a novel substrate, a protein of 130 KDa (p130) but also, the ubiquitin mediated proteolysis of SHP-1, a phosphoprotein of 65 KDa with an activity of tyrosine phosphatase that constitutes a negative regulator of signaling induced by the system SCF/c-kit^{WT}. The differences observed between the two forms of c-kit suggest that the signals transduced by c-kit^{WT} stimulated by SCF and by c-kit^{D814Y} are not equivalent. A precise analysis of intracellular messengers recruited by normal or mutated c-kit could lead to the discovery of new therapeutic alternatives that will aim at blocking specially the aberrant signaling pathways.

Finally, some genetic modifications of c-kit can alter the internalization signal with, as a consequence, a prolonged activation of c-kit. In fact, the c-kit^{d(573-579)} is not or little internalized in the absence of SCF, whereas the activated c-kit^{D814V} receptor is continuously degraded, even in the absence of SCF (Moriyama et al, J Biol Chem. 271: 3347-50, 1996).

1. A method for treating mastocytosis comprising administering a tyrosine kinase inhibitor to a mammalian in need of such treatment, wherein said inhibitor is unable to promote death of IL-3 dependent cells cultured in presence of IL-3.

2. A method according to claim 1, wherein said tyrosine kinase inhibitor is a non-toxic, selective and potent c-kit inhibitor.

3. A method according to claim 2, wherein said inhibitor is selected from the group consisting of indolinones, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, seleoindoles, selenides, tricyclic polyhydroxylic compounds and benzylphosphonic acid compounds.

4. A method for treating mastocytosis comprising administering a non toxic, potent and selective c-kit inhibitor to a mammalian in need of such treatment, selected from the group consisting of:
pyrimidine derivatives, more particularly N-phenyl-2-pyrimidine-amine derivatives.
indolinone derivatives, more particularly pyrrol-substituted indolinones,
monocyclic, bicyclic aryl and heteroaryl compounds,
and quinazoline derivatives,
wherein said inhibitor is unable to promote death of IL-3 dependent cells cultured in presence of IL-3.

5. A method according to claim 4, wherein said inhibitor is an inhibitor of activated c-kit selected from a constitutively activated-mutant c-kit and/or SCF-activated c-kit.

6. A method according to claim 5, wherein the activated-mutant c-kit has at least one mutation selected from mutations proximal to Y823, more particularly between amino acids 800 to 850 of SEQ ID No1 involved in c-kit autophosphorylation, notably the D816V, D816Y, D816F and D820G mutants, and a deletion in the juxtamembrane domain of c-kit, preferably between codon 573 and 579.

7. A method for treating mastocytosis comprising administering to a mammalian in need of such treatment a compound that is a selective, potent and non toxic inhibitor of activated c-kit obtainable by a screening method which comprises:
a) bringing into contact (i) activated c-kit and (ii) at least one compound to be tested; under conditions allowing the components (i) and (ii) to form a complex,
b) selecting compounds that inhibit activated c-kit,
c) testing and selecting a subset of compounds identified in step b), which are unable to promote death of IL-3 dependent cells cultured in presence of IL-3.

8. A method according to claim 7, wherein the screening method further comprises the step consisting of testing and selecting a subset of compounds identified in step b) that are inhibitors of mutant activated c-kit, which are also capable of inhibiting SCF-activated c-kit wild.

9. A method according to claim 7, wherein activated c-kit is SCF-activated c-kit wild.

10. A method according to one of claims 7 to 9, wherein putative inhibitors are tested at a concentration above 10 μM in step a).

11. A method according to one of claims 7 to 10, wherein IL-3 is present in the culture media of IL-3 dependent cells at a concentration comprised between between 0.5 and 10 ng/ml, preferably between 1 to 5 ng/ml.

12. A method according to one of claims 7 to 11, wherein the extent to which component (ii) inhibits activated c-kit can be measured in vitro or in vivo.

13. A method according to one of claims 7 to 12 wherein, the screening method further comprises the step consisting of testing and selecting in vitro or in vivo compounds capable of inhibiting c-kit wild at concentration below 1 μM.

14. A method according to claim 13 wherein, wherein the test is performed using cells lines selected from the group consisiting of mast cells, transfected mast cells, BaF3, and IC-2.

15. A method according to claim 13 wherein, wherein the test includes the determination of the amount of c-kit phosphorylation.

16. A method for treating mastocytosis according to one of claims 7 to 12, wherein the screening comprises:
a) performing a proliferation assay with cells expressing a mutant c-kit (for example in the transphosphorylase domain), which mutant is a permanent activated c-kit, with a plurality of test compounds to identify a subset of candidate compounds targeting activated c-kit, each having an IC50<10 μM, by measuring the extent of cell death,
b) performing a proliferation assay with cells expressing c-kit wild said subset of candidate compounds identified in step (a), said cells being IL-3 dependent cells cultured in presence of IL-3, to identify a subset of candidate compounds targeting specifically c-kit,
c) performing a proliferation assay with cells expressing c-kit, with the subset of compounds identified in step b) and selecting a subset of candidate compounds targeting c-kit wild, each having an IC50<10 μM, preferably an IC50<1 μM, by measuring the extent of cell death.

17. A method according to one of claims 1 to 16 for treating category I, II, III and IV mastocytosis in human and any symptom associated with category I, II, III and IV mastocytosis.

18. A method according to claim 17 for treating urticaria pigmentosa, diffuse cutaneous mastocytosis, solitary mastocytoma in human, bullous, erythrodermic and teleangiectatic mastocytosis.

19. A method according to claim 18, wherein the inhibitor is administered topically.

20. A method according to claim 19, wherein a dermatological composition comprising the inhibitor is applied to the skin.

21. A method according to claim 17 for treating mastocytosis with an associated hematological disorder, such as a myeloproliferative or myelodysplastic syndrome, acute leukemia, myeloproliferative disorder associated with mastocytosis, and mast cell leukemia.

22. A method according to one of claims 1 to 16 for treating dog mastocytoma.

23. A composition for topical application comprising a tyrosine kinase inhibitors, more particularly a non toxic, potent and selective c-kit inhibitor.

24. A composition according to claim 23, which is suitable for topical application.

25. A composition according to claim 24, which is in the form of a gel, paste, ointment, cream, lotion, liquid suspension aqueous, aqueous-alcoholic or, oily solutions, or dispersions of the lotion or serum type, or anhydrous or lipophilic gels, or emulsions of liquid or semi-solid consistency of the milk type, obtained by dispersing a fatty phase in an aqueous phase or vice versa, or of suspensions or emulsions of soft, semi-solid consistency of the cream or gel type, or alternatively of microemulsions, of microcapsules, of microparticles or of vesicular dispersions to the ionic and/or nonionic type.

26. A composition according to claim 25, which comprises at least one ingredient selected from hydrophilic or lipophilic gelling agents, hydrophilic or lipophilic active agents, emollients, viscosity enhancing polymers, humectants, surfactants, preservatives, antioxidants, solvents, and fillers.

27. Use of a composition according to one of claims 23 to 26 for treating skin disorders in human associated with mastocytosis, notably cutaneous mastocytosis including urticaria pigmentosa, diffuse cutaneous mastocytosis, solitary mastocytoma and bullous, erythrodermic and teleangiectatic mastocytosis.

28. Product comprising at least one tyrosine kinase inhibitor, preferably a c-kit inhibitor, wherein said inhibitor is unable to promote death of IL-3 dependent cells cultured in presence of IL-3 and at least one compound selected from 2-Chloro-2′-desoxyadenosine and analogs thereof for a separate, sequential or simultaneous use for treating category IV mastocytosis including mast cell leukemia.

29. Product comprising at least one tyrosine kinase inhibitor, preferably a c-kit inhibitor, wherein said inhibitor is unable to promote death of IL-3 dependent cells cultured in presence of IL-3 and IFNα for a separate, sequential or simultaneous use for treating systemic forms of mastocytosis, especially category III mastocytosis.
